(19) United States
(12) Patent Application Publication
(10) Pub. No.: US 2025/0114192 A1
(43) Pub. Date: Apr. 10, 2025

(54) VALVE SKIRTS FOR PROSTHETIC DEVICES

(71) Applicant: Medtronic, Inc., Minneapolis, MN (US)

(72) Inventors: Yogesh Darekar, Irvine, CA (US); Brenda McIntire, Santa Rosa, CA (US); Jason Fox, Centennial, CO (US); Kyle Kenyon, Irvine, CA (US)

(21) Appl. No.: 18/730,946

(22) PCT Filed: Jan. 20, 2023
(86) PCT No.: PCT/IB2023/050498

§ 371 Date: Jul. 22, 2024

Related U.S. Application Data
(60) Provisional application No. 63/267,262, filed on Jan. 28, 2022.

(51) Int. Cl. A61F 2/24

(52) U.S. Cl. CPC A61F 2/2418; A61F 2/2415; A61F 2220/0075

(57) ABSTRACT

Valve skirts for prosthetic devices are provided. In particular, valve skirts for attachment to an outer frame of a heart valve prosthesis are provided. Valve skirts provided herein include one piece valve skirts. One piece valve skirts may be configured to reduce a size of a prosthetic heart valve in a reduced diameter delivery configuration.

### FIELD

The present technology is generally related to heart valve prostheses implantable via minimally invasive procedures, and in particular is directed to valve skirts for mitral valve prostheses.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and shut in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems, and are delivered in a radially compressed or crimped configuration for advancement through the patient's vasculature. Accordingly, once positioned at a treatment site, a heart valve prosthesis may be expanded, or permitted to return to an uncompressed state, to engage tissue at the diseased heart valve region to, for instance, hold the heart valve prosthesis in position.

While these valve prostheses offer minimally invasive methods for heart valve repair and/or replacement, challenges remain such as reducing the size of a heart valve prosthesis during delivery procedures. Heart valve prosthesis delivery is frequently achieved via catheter, with the heart valve prosthesis being delivered in a reduced-diameter delivery configuration. Minimizing the size of the reduced-diameter heart valve prosthesis may serve to facilitate or ease the delivery process. Therefore, there exists a need for size reduction of heart valve prosthesis in their reduced-diameter delivery configuration.

The present disclosure relates to improvements in valve skirt designs configured to reduce the overall size of a heart valve prosthesis when in a reduced-diameter delivery configuration.

### SUMMARY

The present disclosure relates to improvements in valve skirt designs configured to reduce the overall size of a heart valve prosthesis when collapsed.

In an embodiment, a heart valve prosthesis is provided. The heart valve prosthesis includes a frame including an inner frame configured to support a prosthetic valve component, and an outer frame coupled to the inner frame, the outer frame being sized to surround the inner frame and configured to anchor the heart valve prosthesis, the outer frame including a plurality of first crowns positioned around an outflow end of the outer frame and a plurality of second crowns positioned inward of the plurality of first crowns; and a skirt disposed within and coupled to the outer frame and consisting of a single piece of material.

In an embodiment, a method of assembling a heart valve prosthesis including an inner frame, an outer frame disposed around the inner frame, a valve component supported by the inner frame, and a skirt consisting of a single piece of material is provided. The method includes forming the skirt into a three-dimensional configuration by securing a plurality of connection edges to one another; disposing the skirt within the outer frame, wherein a ring portion of the skirt including an inflow edge corresponds to a ring section of the outer frame and a conical portion of the skirt including an outflow edge corresponds to a tapered portion of the outer frame; and coupling the skirt to the outer frame.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the art to make and use the invention. The drawings are not to scale.

FIG. 1 depicts a perspective view of a heart valve prosthesis in accordance with an aspect of this disclosure.

FIG. 2 depicts a ventricle, or bottom, outflow view of the heart valve prosthesis of FIG. 1 in accordance with an aspect of this disclosure.

FIG. 3 depicts a side view of a heart valve prosthesis employing a two-piece outer skirt.

FIG. 4 illustrates a one-piece outer skirt in a lay flat configuration, according to embodiments hereof.

FIGS. 5A and 5B illustrate a one piece outer skirt as attached to a heart valve prosthesis, according to embodiments hereof.

FIG. 6 illustrates a one-piece outer skirt in a lay flat configuration, according to embodiments hereof.

FIG. 7 illustrates a further embodiment of a one-piece outer skirt in a lay flat configuration, according to embodiments hereof.

FIG. 8 illustrates a one-piece outer skirt in a lay flat configuration, according to embodiments hereof.

FIGS. 9A and 9B illustrate a one piece outer skirt as attached to a heart valve prosthesis, according to embodiments hereof.

FIG. 10 illustrates a method of assembling a prosthetic heart valve consistent with embodiments hereof.

### DETAILED DESCRIPTION

Specific embodiments of the present invention are now described with reference to the figures. The terms “inflow” and “outflow”, when used in the following description refer to a native vessel, native valve, or a device to be implanted into a native vessel or native valve, such as a heart valve prosthesis, are with reference to the direction of blood flow. Thus, “inflow” refers to positions in an upstream direction with respect to the direction of blood flow and the term “outflow” refers to positions in a downstream direction with respect to the direction of blood flow.

The following detailed description is merely exemplary in nature and is not intended to limit the invention or the application and uses of the invention. Although the description of embodiments hereof is in the context of the treatment of heart valves such as the pulmonary, aortic, mitral, or tricuspid valve, the invention may also be used in other body passageways where it is deemed useful. Furthermore, there is no intention to be bound by any expressed or implied theory presented in the preceding technical field, background, brief summary or the following detailed description.

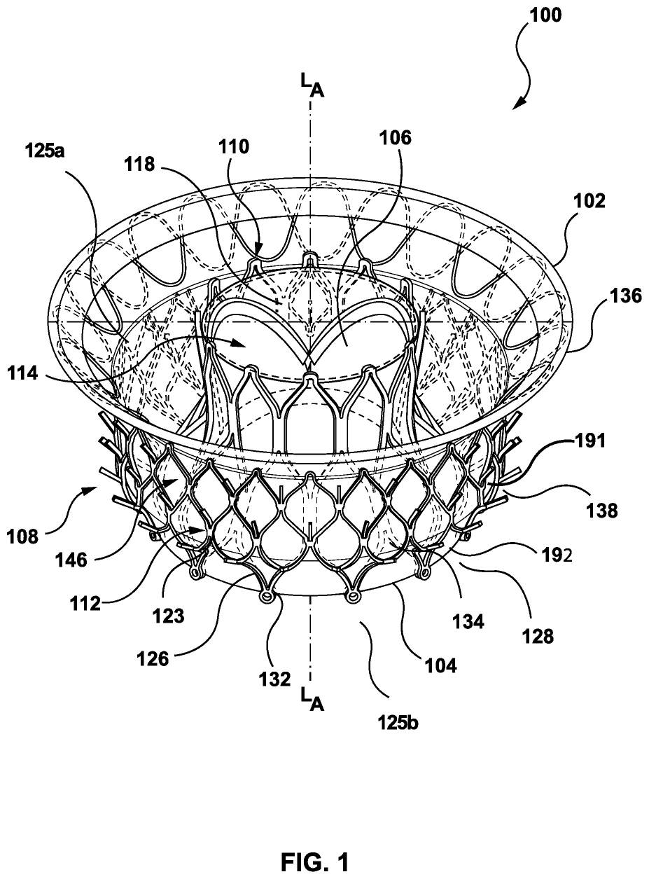

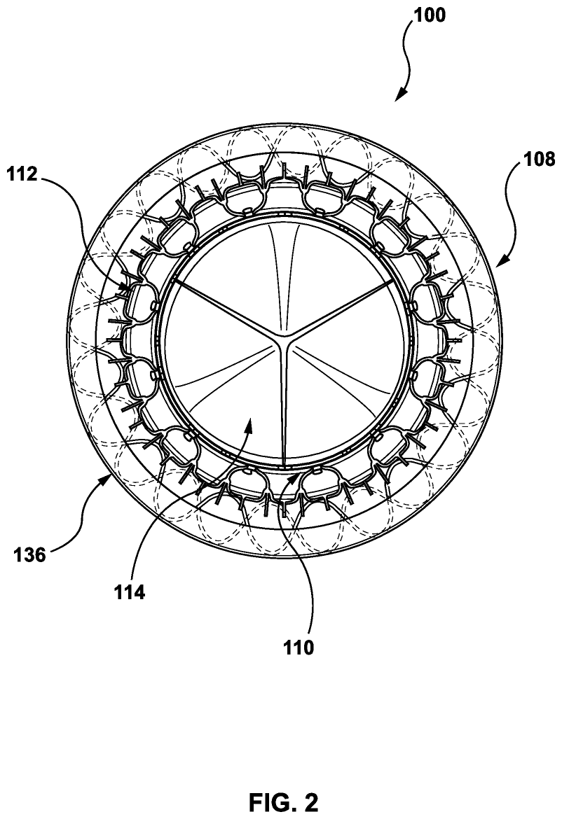

A perspective view of a transcatheter heart valve prosthesis 100 in accordance with an aspect of the disclosure is shown in FIG. 1, with FIG. 2 depicting a bottom or outflow view of the heart valve prosthesis 100. The heart valve prosthesis 100 is configured to be compressed into a reduced-diameter delivery configuration within a delivery catheter and to return to an expanded, deployed configuration when delivered/released from the delivery catheter within a native mitral valve. In some embodiments, the heart valve prosthesis 100 includes a frame 108, a brim element 136 secured to the frame, and a prosthetic valve component 114. The frame 108 has a stent-like structure that is configured to support the prosthetic valve component 114 and to define, along a longitudinal axis LA thereof, a blood flow lumen 106 that substantially extends from an inflow end 102 to an outflow end 104 of the heart valve prosthesis 100. In some embodiments, the frame 108 generally includes an inner frame 110 and an outer frame 112. In aspects hereof, the inner frame 110 of the frame 108 may be alternatively referred to as a valve support element, an inner portion, or valve housing. The outer frame 112 of the frame 108 may be alternatively referred to as an anchoring element, a fixation ring, or an outer frame. The inner frame 110 is configured to hold the prosthetic valve component 114, and the outer frame 112, which surrounds the inner frame 110, is configured to secure the heart valve prosthesis 100 to the native tissue of the heart when implanted in vivo. The frame 108 may be considered to have a dual-stent structure.

The inner frame 110 includes a stent-like structure supporting a prosthetic valve component 114 capable of regulating flow therethrough. When deployed in situ, the prosthetic valve component 114 in a closed state is configured to block blood flow in one direction to regulate blood flow through a blood flow lumen 106 of the inner frame 110. The inner frame 110 further includes an inner skirt 118 configured to permit blood to flow from the atrium to the ventricle through the blood flow lumen 106 of the prosthetic valve component 114 when it is in an open state.

The outer frame 112 is configured to secure the heart valve prosthesis 100 to the native valve and the surrounding sub annular tissue. The outer frame 112 includes a stent-like structure comprising a plurality of cells 134 defined by a plurality of struts 126 and nodes 123 of the outer frame 112. At the outflow end 125b of the outer frame 112, a plurality of crowns 132 are formed by respective pairs of opposing struts 126.

The outer frame 112 is positioned around the inner frame 110 and defines an inflow end 125a of the outer frame 112 having a first diameter and an outflow end 125b of the outer frame 112 having a second diameter that is smaller than the first diameter. The outer frame 112 includes a ring section 138 having a substantially cylindrical profile. In the ring section 138, the diameter of the outer frame 112 is the first diameter. The outer frame 112 further includes a tapered section 128. In the tapered section 128, the diameter of the outer frame 112 transitions from the first diameter to the second diameter and therefore is tapered or conical in shape. At least a portion of the outer surface of the outer frame 112, when the heart valve prosthesis 100 is in an expanded state, is configured to be disposed against the native tissue of the heart for securing the outer frame 112 and, concurrently, the heart valve prosthesis 100.

Further, the outer frame 112 is mechanically isolated from the inner frame 110. The outer frame 112 may deform upon implantation within a native mitral valve annulus, and/or expand and contract in response to movement of the native tissue, while remaining spaced from the inner frame 110, which thereby permits the inner frame 110 to remain relatively still and undeformed. The inner frame 110 is, therefore, isolated from external forces, allowing for the prosthetic valve component 114 to more efficiently replicate the function of the native mitral valve.

In accordance with aspects hereof, the inner frame 110 and the outer frame 112 of the frame 108 of the heart valve prosthesis 100 may be made from any number of suitable biocompatible materials, e.g., stainless steel, nickel titanium alloys such as Nitinol™, cobalt chromium alloys such as MP35N, other alloys such as ELGILOY® (Elgin, Ill.), various polymers, pyrolytic carbon, silicone, polytetrafluoroethylene (PTFE), or any number of other materials or combination of materials. A suitable biocompatible material would be selected to provide the heart valve prosthesis 100 to be configured to be compressed into a reduced diameter configuration for transcatheter delivery to a native valve, whereby release from a delivery catheter allows the prosthesis 100 to self-expand, returning to an expanded, deployed configuration. In some embodiments, the self-expansion is accomplished through the use of a shape-memory material such as Nitinol™. The heart valve prosthesis 100 may be processed to have a default or “set” shape that coincides with the deployed configuration. Therefore, once the compressed heart valve prosthesis 100 is delivered and released, the prosthesis 100 will return to the default or “set” deployed configuration.

The heart valve prosthesis 100 may further include a brim or preshaped wire element 136 that extends outwardly from the inflow end 125a of the outer frame 112. The brim 136 may include overlapping, 180 degree out of phase sinusoidal wire forms that are attached and hinged to the outer frame 112 by a suitable biocompatible low-profile fabric used in bioprosthetic implants namely endovascular grafts, valves or left atrial appendage devices to promote bio-integration, such as woven polyethylene terephthalate (PET) fabric. The brim element 136 may act as an atrial retainer, if present, and to serve such a function the brim element 136 may be configured to engage tissue above a native annulus, such as a supra-annular surface or some other tissue in the left atrium, to thereby inhibit downstream migration of the heart valve prosthesis 100 as well as mitigate any leakage through any gaps between native tissue and the brim, for e.g., during atrial systole.

An inner skirt 118 is disposed within and is coupled to the inner frame 110, and, more particularly, is coupled as to line an inner surface of the inner frame 110, or at least a substantial portion thereof. The inner skirt 118 extends from the inflow end of the inner frame 110 to the outflow end of the inner frame 110. When the heart valve prosthesis 100 is implanted within a native mitral valve, the inner skirt 118 is configured to limit the amount of unintentional blood leakage, otherwise known as regurgitation, between a left atrium and a left ventricle. The inner skirt 118 covers the inner surface of the inner frame 110, as to only allow blood to flow from the atrium to the ventricle when the prosthetic valve component 114 is in the open state. The inner skirt 118 may take the form of a single piece or multiple pieces of material that is wrapped within the inner surface so as to create a cylindrical body that is flush with the inner surface. The inner skirt 118 is then coupled to the inner frame 110 using sutures or adhesive.

An outer skirt 146 is disposed within and is coupled to the outer frame 112. The outer skirt 146 extends from the inflow end 125a of the outer frame 112 to the outflow end 125b of the outer frame 112. The outer skirt 146 is configured to match the both the cylindrical profile of the ring section 138 and the tapered or conical profile of the tapered section 128.

When the heart valve prosthesis 100 is implanted within a native mitral valve, the outer skirt 146 substantially covers the inner surface of the outer frame 112 so as to limit unintentional blood flow from the left atrium to the left ventricle. The outer skirt 146 is wrapped within the inner surface as to create a shape that is flush with the outer frame 112. The outer skirt 146 is affixed to the outer frame 112 using sutures or adhesive. To inhibit blood flow, the outer skirt 146 is further configured to substantially cover the cells 134 of the outer frame 112.

As shown in FIG. 1 the outer skirt 146 has a ring portion 191 corresponding to the ring section 138 of the outer frame 112 and a conical portion 192 corresponding to the tapered section 128 of the outer frame 112. The outer skirt 146, configured to be disposed flush with the outer frame 112 and therefore also tapers in the conical portion 192 to match the tapered section 128 of the frame.

In embodiments, the inner skirt 118 and the outer skirt 146 as well as all other valve skirts discussed herein, may be manufactured, e.g., cut from, a flat material. In embodiments hereof, the inner skirt 118 and the outer skirt 146 may be formed of a low-porosity woven fabric, such as polyester, Dacron fabric, or PTFE. In further aspects, the inner and outer skirts 182, 146 may be a knit polyester, such as a polyester or PTFE knit, which can be used when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. In further aspects hereof, the inner and outer skirts 118, 146 may be formed of a natural or biological material such as pericardium or another membranous tissue such as intestinal submucosa. Further, in embodiments, the inner skirt 118 and the outer skirt 146 may be made of the same or different materials, for example, the inner skirt 118 may be made from a PTFE knit, while the outer skirt 146 is made of a woven polyester.

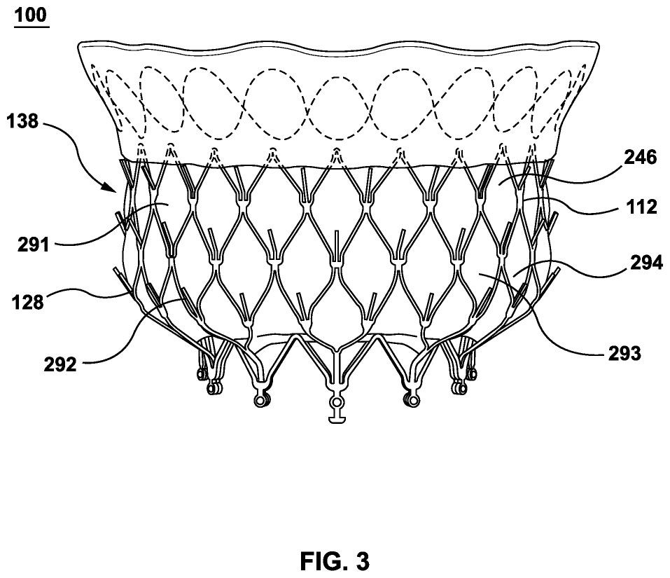

Conforming a flat material to a three-dimensional structure may present difficulties, potentially resulting in excess skirt volume, bulges, tents, and other imperfections in the fit between the outer skirt 146 and the outer frame 112. Such imperfections in the fit may result in a larger size of the overall heart valve prosthesis 100 when in a reduced-diameter configuration. Accordingly, as discussed below with respect to FIGS. 3-9B, various solutions are provided to reduce or minimize the size of the heart valve prosthesis 100 in the reduced-diameter configuration through improvements to the design and shape of the outer skirt 146.

FIG. 3 illustrates the heart valve prosthesis 100 employing a two piece outer skirt 246. The two piece outer skirt 246 includes two pieces, a ring portion 291 and a conical portion 292. The ring portion 291 and the conical portion 292 are connected to one another along an overlapping portion 293 and secured to one another via a suture line 294. The two piece outer skirt 246 is secured to the outer frame 112 of the heart valve prostheses 100, e.g., via one or more sutures. The two piece outer skirt 246 uses two pieces having different shapes to achieve conformity with the ring section 138 and the tapered section 128 of the outer frame 112. The two piece outer skirt 246 may have the drawback of added complexity relative to one piece solutions (discussed below) and may further have the drawback of additional material usage, due to the necessity of an overlapping portion 293 to secure the ring portion 291 and the conical portion 292. The additional material from the overlapping portion 293 may cause an increased diameter and/or an increased packing density when the heart valve prosthesis 100 is in the reduced diameter delivery configuration.

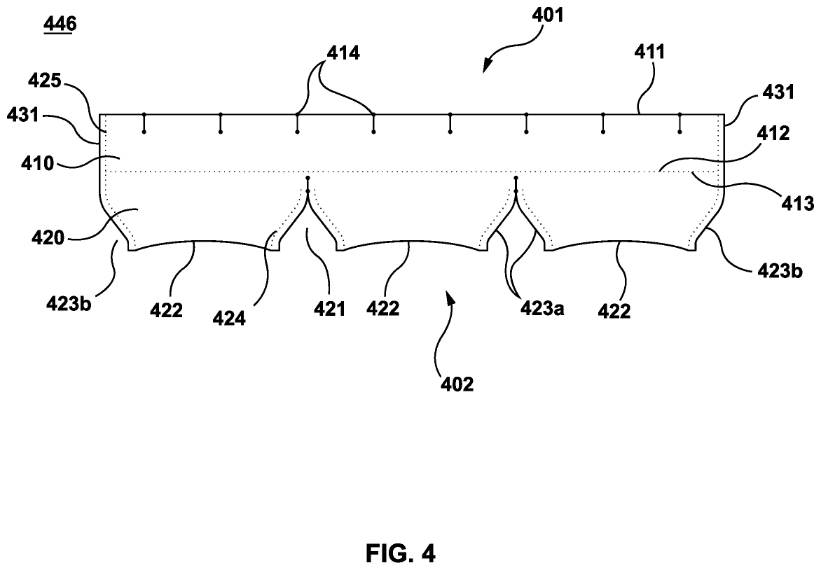

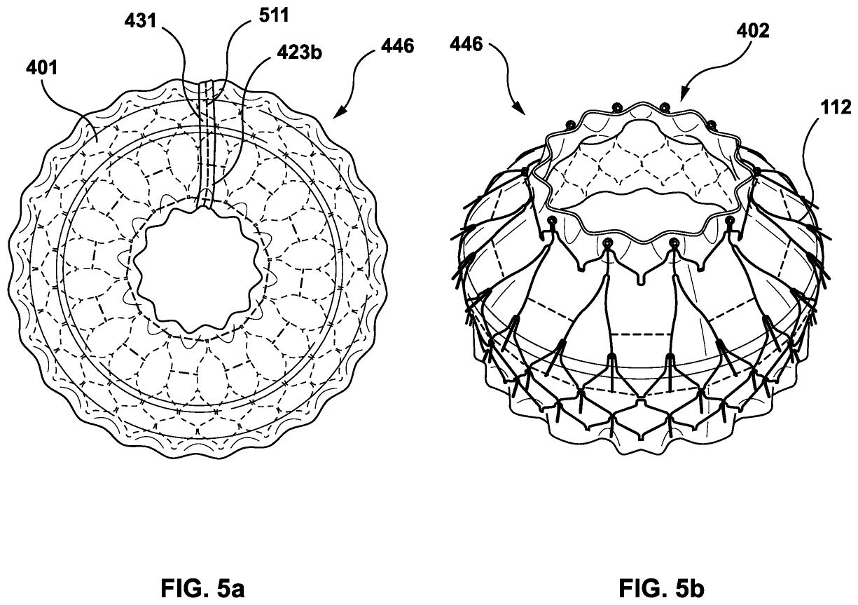

FIGS. 4, 5A, and 5B illustrates an example embodiment of a one-piece outer skirt. FIG. 4 illustrates the one-piece outer skirt 446 in a lay flat configuration while FIGS. 5A and 5B show the one-piece outer skirt 446 after attachment to the outer frame 112 of the heart valve prosthesis 100.

FIG. 4 illustrates the one-piece outer skirt 446 in a lay flat configuration. The one piece outer skirt 446 includes a ring portion 410 and a conical portion 420. The one piece outer skirt 446 further includes an inflow end 401 and an outflow end 402. The outflow end 402 is disposed at an end of the conical portion 420 opposite the ring portion 410 and corresponds to the outflow end 125b of the outer frame 112. The inflow end 401 is disposed at an end of the ring portion 410 opposite the conical portion 420 and corresponds to the inflow end 125a of the outer frame 112.

The ring portion 410 is substantially rectangular, with an inflow edge 411 corresponding to the inflow end 401, two ring connection edges 431 disposed at opposite short sides of the ring portion 410, and a central boundary 412 that is shared with the conical portion 420. The central boundary 412 represents a boundary between the ring portion 410 and the conical portion 420, but is not a true edge of the material from which the one piece outer skirt 446 is constructed. Accordingly, the one piece outer skirt 446 is continuous across the central boundary 412. The central boundary 412 may include one or more central suture holes 413. The central suture holes 413 are disposed along the central boundary 412 and may be used for attaching the one piece outer skirt 446 to the outer frame 112 via one or more sutures. The one piece outer skirt 446 may be sutured to the outer frame 112 via sutures through the suture holes 413 and/or directly through the material of the outer skirt 446 and attached to the outer frame 112 frame, for example, at either nodes 123 or struts 126 of the outer frame 112.

In embodiments, the ring portion 410 includes one or more slits 414 disposed at the inflow edge 411. The slits 414 may be configured to facilitate attachment between the one piece outer skirt 446 and the valve brim 136. The valve brim 136 may be larger in diameter than the ring portion 410 of the outer skirt 446. The slits 414 may therefore provide additional flexibility to facilitate attachment to the valve brim 136.

The conical portion 420 is configured to form a tapered or conical section of the one piece outer skirt 446, providing a tapered transition from an inflow diameter corresponding to the first diameter of the outer frame 112 to an outflow diameter corresponding to the second diameter of the outer frame 112. The inflow diameter is larger than the outflow diameter. In the lay flat configuration of FIG. 4, the conical portion 420 includes a discontinuous outflow edge 422, interrupted by a plurality of notches 421. Spanning the gaps in the outflow edge 422 are notch edges 423. The notch edges 423 include inner notch edges 423a and outer notch edges 423b. The inner notch edges 423a are disposed opposing a corresponding inner notch edge 423a when the outer skirt 446 is in a lay flat configuration. The outer notch edges 423b are disposed at either end of the outer skirt 446 in the lay flat configuration and are configured to meet each other when the outer skirt 446 is formed into a three-dimensional configuration. The length of the discontinuous outflow edge 422 corresponds to a circumference of the outflow diameter of the outer skirt 446

The one piece outer skirt 446 is configured to be formed into a three-dimensional configuration by securing the opposing ring connection edges 431 to one another and by securing opposing inner notch edges 423a to one another and the outer notch edges 423b to one another. The ring connection edges 431 are secured to one another via ring edge suture holes 425. In embodiments, the ring edge suture holes 425 may also be used to facilitate a sutured attachment of the ring portion 410 to the outer frame 112, e.g., via the nodes 123 or the struts 126. The opposing notch edges 423a, disposed opposite one another at corresponding notches 421, may be sutured together through one or more notch suture holes 424 disposed along the inner notch edges 423a. The outer notch edges 423b disposed at opposite ends of the one piece outer skirt 446 when it is in a lay flat configuration are also secured to one another when the one piece outer skirt 446 is formed into a three-dimensional configuration. The conical portion 420 is configured such that, when the one piece outer skirt 446 is formed into a three-dimensional configuration, the opposing inner notch edges 423a and the outer notch edges 423b meet and the outflow edge 422 is substantially continuous and defines the outflow diameter of the outer skirt 446. In embodiments, the notch suture holes 424 may also be used to facilitate a sutured attachment of the conical portion 420 to the outer frame 112, e.g., via the nodes 123 or the struts 127.

FIGS. 5A and 5B illustrate an outer frame 112 of the heart valve prosthesis 100 with the one piece outer skirt 446 attached thereto. The outer notch edges 423b are secured to one another and the ring connection edges 431 are secured to one another, creating a seam 511. The seam 511 and the seams (not shown) where the opposing inner notch edges 423a are the only seams in the outer skirt 446 in the three-dimensional configuration, which minimizes the amount of excess material consumed by the overlap required at such seams. These seams run parallel to the axis of the outer skirt 446 in the three-dimensional configuration and are relatively short compared to the perpendicular seams creating the overlapping portion 293 around the full diameter of the outer skirt 146, as discussed above. FIG. 5A is shown from the inflow end 401 of the outer skirt 446 and FIG. 5B is shown from the outflow end 402 of the outer skirt 446. As shown in FIGS. 5A and 5B, when attached to the outer frame 112, the outer skirt 446 may have no or minimal overlap with itself or another piece of skirt material when the heart valve prosthesis is in the expanded configuration. For example, the amount of overlap may range between 0% and 5% or between 2% and 5% of the total valve skirt area.

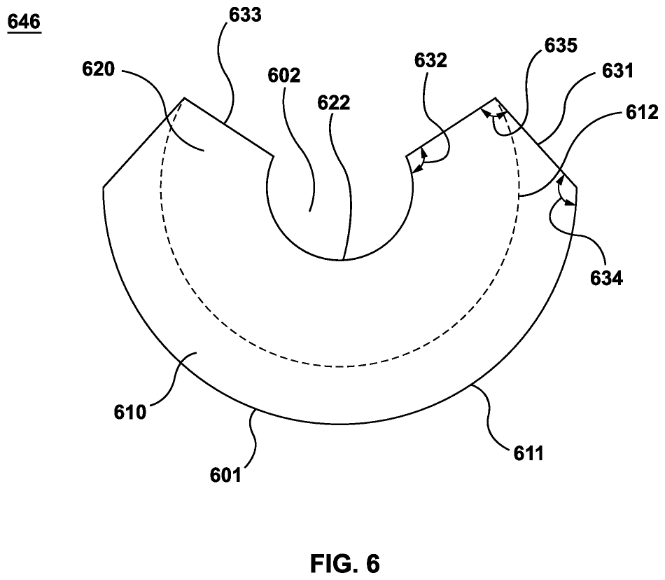

FIG. 6 illustrates an example embodiment of a one-piece outer skirt in a lay flat configuration. The one piece outer skirt 646 includes a ring portion 610 and a conical portion 620. The one piece outer skirt 646 further includes an inflow end 601 and an outflow end 602. The outflow end 602 is disposed at an end of the conical portion 620 opposite the ring portion 610 and corresponds to the outflow end 125b of the heart valve prosthesis 100. The inflow end 601 is disposed at an end of the ring portion 610 opposite the conical portion 420 and corresponds to the inflow end 125a of the heart valve prosthesis 100.

The ring portion 610 is formed from an annular section of material, with an inflow edge 611 representing an arc section of a circle of a first diameter corresponding to the inflow end 601 and a central boundary 612, representing an arc section of a circle concentric with the inflow edge 611. The length of the inflow edge 611 corresponds to an inflow diameter of the outer skirt 646 when assembled in a three-dimensional configuration and further corresponds to the first diameter of the outer frame 112. The central boundary 612 is shared with the conical portion 620. The central boundary 612 represents a boundary between the ring portion 610 and the conical portion 620, but is not a true edge of the material from which the one piece outer skirt 646 is constructed. Accordingly, the one piece outer skirt 646 is continuous across the central boundary 612.

The ring portion 610 further includes two ring connection edges 631 connecting the inflow edge 611 to the central boundary 612. The ring connection edges 631 form angles 634 with the inflow edge 611. The angles 634 between the ring connection edges 631 and the inflow edge 611 are obtuse, greater than 90°. Thus, the ring connection edges 631 are not collinear with radii of the arcs represented by the inflow edge 611 and the central boundary 612. The ring portion 610 is configured to form a cylindrical section of the one piece outer skirt 646 corresponding to the ring section 138 of the outer frame 112 when the outer skirt 646 is in a three-dimensional configuration through attachment of the two ring connection edges 631.

The conical portion 620 is formed from an annular section of material, with an outflow edge 622 representing an arc section of a circle of an outflow diameter corresponding to the outflow end 602 and a central boundary 612, representing an arc section of a circle concentric with the outflow edge 622. The outflow diameter corresponds with the second diameter of the outer frame 112. The central boundary 612 is shared with the ring portion 610.

The conical portion 620 is configured to form a tapered or conical section of the one piece outer skirt 646 corresponding with the tapered section 128 of the outer frame 112 when the one piece outer skirt 646 is in a three-dimensional configuration. The conical portion 620 provides a tapered transition from a larger diameter corresponding to the first diameter of the outer frame 112 to a smaller diameter corresponding to the second diameter of the outer frame 112. In the lay flat configuration of FIG. 6, the conical portion 620 includes an outflow edge 622 and shares the central boundary 612 with the ring portion 610. The outflow edge 622 and the central boundary 612 are arc sections of circles sharing the same center. Accordingly, the conical portion 610 represents an annular section.

The outflow edge 622 and the central boundary 612 are joined by the cone connection edges 633, which meet the ring connection edges 631 at the central boundary 612. The cone connection edges 633 may meet the ring connection edges 631 at angle 635. The cone connection edges 633 extend from the outflow edge 622 at angle 632. In embodiments, the angles 632, 634, and 635 may vary depending on a size and geometry of the outer frame 112. In embodiments, the angles 632 and 634 may vary between approximately 90 and approximately 135 degrees. In embodiments, the angle 635 may vary between approximately 70 and 120 degrees.

The one piece outer skirt 646 is configured to be formed into a three-dimensional configuration by securing the opposing ring connection edges 631 to one another and by securing opposing cone connection edges 633 to one another. In the three-dimensional configuration, the one piece outer skirt 646 is secured to the outer frame 112 of the heart valve prosthesis 100, e.g., via the nodes 123 or struts 126.

In embodiments, the central boundary 612, the ring connection edges 631, and/or the cone connection edges 633 may include one or more suture holes to facilitate assembly of the outer skirt 646 and attachment of the outer skirt 646 to the outer frame 112. The one piece outer skirt 646 may be sutured to the outer frame 112 via sutures through such suture holes and/or directly through the material of the outer skirt 646 and attached to the outer frame 112 frame. When attached to the outer frame 112, the outer skirt 646 may have no or minimal overlap with itself or another piece of skirt material when the heart valve prosthesis is in the expanded configuration. For example, the amount of overlap may range between 0% and 5% or between 2% and 5% of the total valve skirt area.

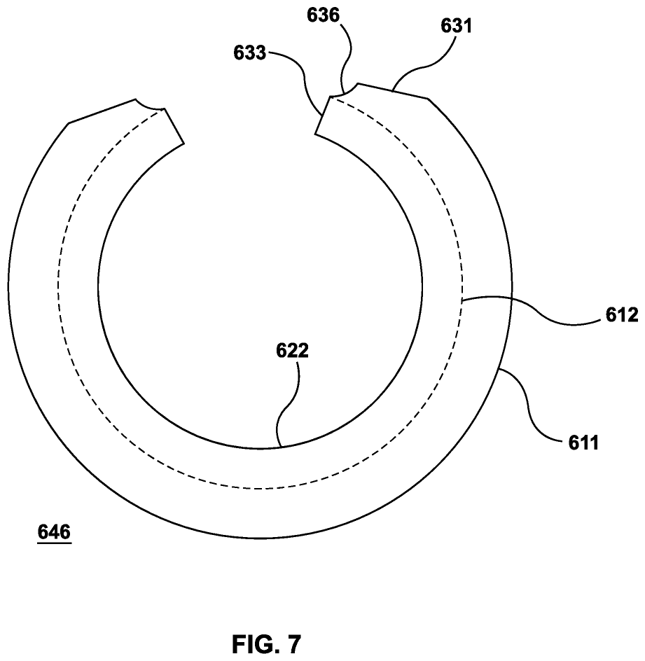

FIG. 7 illustrates an alternate embodiment of the one-piece outer skirt 646 with optional features. In an embodiment, as shown in FIG. 7A, the cone connection edges 633 and the ring connection edges 631 meet at an arcuate edge 636 instead of the angle 634. Each arcuate edge 636 stretches across the central boundary 612 and meets the ring connection edge 631 at one end and the cone connection edge 633 at its other end. The arcuate edge 636 is configured to reduce the amount of tented material when the outer skirt 646 is formed into the three-dimensional configuration thereby reducing the material bulk.

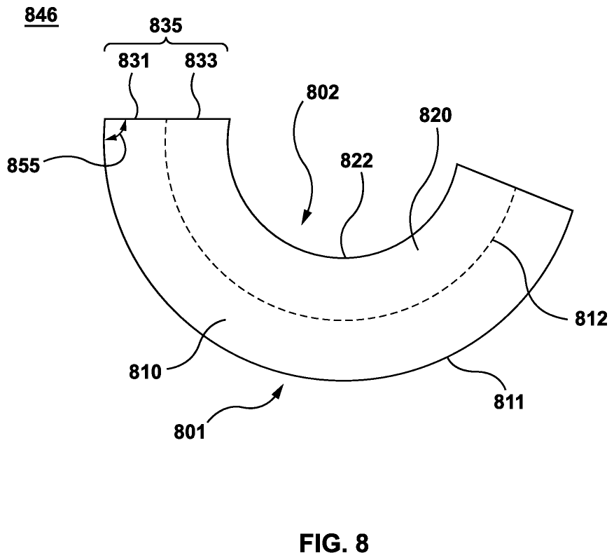

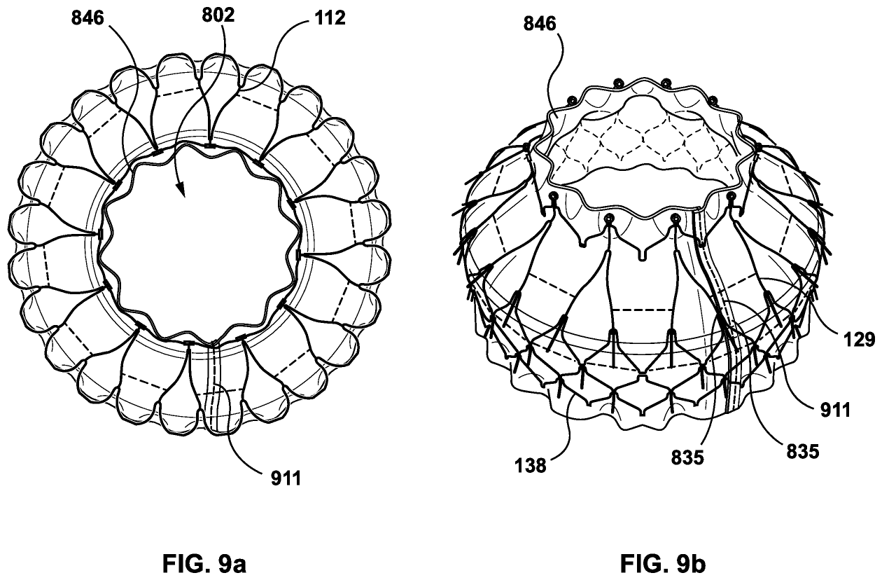

FIGS. 8, 9A, and 9B illustrate an example embodiment of a one-piece outer skirt. FIG. 8 illustrates the one-piece outer skirt in a lay flat configuration while FIGS. 9A and 9B show additional aspects of the one-piece outer skirt after attachment to a heart valve prosthesis outer frame.

FIG. 8 illustrates an example embodiment of a one-piece outer skirt in a lay flat configuration. The one piece outer skirt 846 includes a ring portion 810 and a conical portion 820. The one piece outer skirt 846 further includes an inflow end 801 and an outflow end 802. The outflow end 802 is disposed at an end of the conical portion 820 opposite the ring portion 810 and corresponds to the outflow end 125b of the outer frame 112. The inflow end 801 is disposed at an end of the ring portion 810 opposite the conical portion 820 and corresponds to the inflow end 125a of the outer frame 112.

The ring portion 810 is formed from an annular section of material, with an inflow edge 811 representing an arc section of a circle of a first diameter corresponding to the inflow end 801 and a central boundary 812, representing an arc section of a circle of a second diameter, concentric with the inflow edge 811. The ring portion 810 is configured to form a cylindrical section corresponding to the ring section 138 of the outer frame 112 when in a three-dimensional configuration. The length of the inflow edge 811 corresponds to an inflow diameter of the outer skirt 846 when assembled in a three-dimensional configuration and further corresponds to the first diameter of the outer frame 112. The central boundary 812 is a shared with the conical portion 820. The central boundary 812 represents a boundary between the ring portion 810 and the conical portion 820, but not a true edge of the material from which the one piece outer skirt 846 is constructed. Accordingly, the one piece outer skirt 846 is continuous across the central boundary 812. The ring portion 810 further includes two ring connection edges 831 connecting the inflow edge 811 to the central boundary 812. The ring connection edges 831 form angles 855 with the inflow edge 811.

The conical portion 820 is configured to form a tapered or conical section of the one piece outer skirt 846 when the one piece outer skirt 846 is in a three-dimensional configuration. In the three-dimensional configuration, the conical portion 820 corresponds to the tapered section 128 of the outer frame 112. The conical portion 820 provides a tapered transition from a larger inflow diameter corresponding to the first diameter of the outer frame 112 to a smaller outflow diameter corresponding to the second diameter of the outer frame 112. In the lay flat configuration of FIG. 8, the conical portion 820 includes an outflow edge 822 and shares the central boundary 812 with the ring portion 810. The outflow edge 822 and the central boundary 812 are arc sections of circles sharing the same center. Accordingly, the conical portion 810 represents an annular section.

The outflow edge 822 and the central boundary 812 are joined by the cone connection edges 833, which meet the ring connection edges 831 at the central boundary 812. The cone connection edges 833 are collinear with the ring connection edges 831. This arrangement results in a single continuous connection edge 835 at either short end of the one-piece outer skirt 846. The angle 855 between the connection edges 835 and the inflow edge 811 may be varied to according to the geometry and shape of the outer frame 112 to which it is to be attached. In embodiments, the angle 855 may be an acute angle, a right angle, and/or an obtuse angle.

The one piece outer skirt 846 is configured to be formed into a three-dimensional configuration by securing the opposing connection edges 835, e.g., the ring connection edges 831 and collinear cone connection edges 833, to one another. In the three-dimensional configuration, the one piece outer skirt 846 may then be secured to the outer frame 112 of the heart valve prosthesis 100, e.g., via the nodes 123 or struts 127.

In embodiments, the central boundary 812, the ring connection edges 831, and/or the cone connection edges 833 may include one or more suture holes to facilitate assembly of the outer skirt 846 and attachment of the outer skirt 646 to the outer frame 112. The one piece outer skirt 846 may be sutured to the outer frame 112 via sutures through such suture holes and/or directly through the material of the outer skirt 846 and attached to the outer frame 112 frame.

FIGS. 9A and 9B illustrate the outer frame 112 of the heart valve prosthesis 100 with the one piece outer skirt 846 attached thereto. The connection edges 835 are secured to one another, creating a seam 911. The seam 911 is the only seam in the outer skirt 846 in the three-dimensional configuration, which minimizes the amount of excess material required for the overlap at the seam 911. As shown in FIGS. 9A and 9B, when attached to the outer frame 112, the outer skirt 846 may have no or minimal overlap with itself or another piece of skirt material when the heart valve prosthesis is in the expanded configuration. For example, the amount of overlap may range between 0% and 5% or between 2% and 5% of the total valve skirt area.

Table 1, below, provides example values of total surface areas of the one piece outer skirts 446 and 846 as compared to an example two piece outer skirt. As shown in Table 1, the surface area of the one piece outer skirt 846 is significantly reduced as compared to the example two piece outer skirt. This reduction in surface area leads to a reduction in total skirt volume and thus a reduction in the size of the reduced diameter heart valve prosthesis for delivery purposes. Although the one piece outer skirt 446 does not have a reduced surface area as compared to the example two piece outer skirt, elimination of the overlapping portion 293 as shown in FIG. 3 leads to reduced bulk in the central portion of the heart valve prosthesis when in the reduced diameter configuration for delivery purposes.

TABLE 1

| | | |
|---|---|---|
| One Piece Outer Skirt 446 | 6114.5 | mm\hat{ }2 |
| One Piece Outer Skirt 846 | 5660.6 | mm\hat{ }2 |
| Two Piece Outer Skirt | 6041 | mm\hat{ }2 |

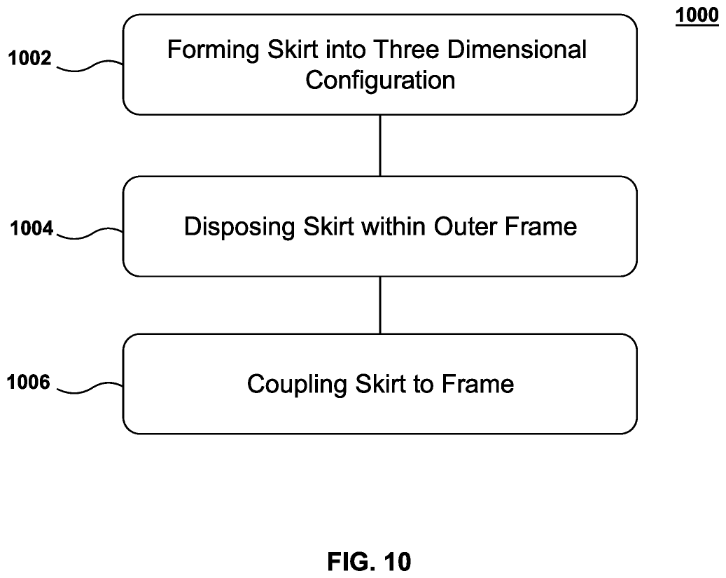

FIG. 10 illustrates a method of assembling a heart valve prosthesis according to embodiments hereof. The method 1000 may be carried out using the heart valve prosthesis 100 and any of the outer skirts (146, 246, 446, 646, 846, etc.) disclosed herein. The method 1000 includes steps in the assembly of a heart valve prosthesis. In embodiments, the method 1000 may include additional steps or fewer steps without departing from the scope of embodiments disclosed herein.

A skirt forming operation 1002 in the method 1000 includes forming an outer skirt into a three-dimensional configuration. As discussed above, outer skirts consistent with embodiments hereof may be manufactured in a flat or two-dimensional configuration from a single piece of material. The outer skirt may be formed into a three-dimensional configuration by securing connection edges (e.g., ring connection edges, conical connection edges, notch edges, etc.) to one another. In the three-dimensional configuration, the outer skirt has a substantially cylindrical ring portion and a tapered conical portion.

A skirt disposition operation 1004 in the method 1000 includes disposing an outer skirt within an outer frame. The outer skirt is disposed within the outer frame. A ring portion of the outer skirt is disposed to correspond to the ring section of the outer frame and a conical portion of the outer skirt is disposed to correspond to a conical section of the outer frame. The outflow edge of the outer skirt is disposed corresponding to the outflow end of the outer frame and the inflow edge of the outer skirt is disposed corresponding to the inflow end of the outer frame.

A skirt coupling operation 1006 in the method 1000 includes coupling or securing to the outer skirt to the outer frame. The outer skirt is coupled to the outer frame via sutures attached, e.g., to both the outer skirt and the outer frame. The sutures may attach or loop around nodes and/or struts of the outer frame and may utilize suture holes preformed in the outer skirt. In some embodiments, new holes may be made in the outer skirt during the suture attachment process. In further embodiments, adhesives may replace or complement the use of sutures.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A heart valve prosthesis comprising:
a frame having expanded and contracted configurations and including
an inner frame configured to support a prosthetic valve component, and
an outer frame coupled to the inner frame, the outer frame being sized to surround the inner frame and configured to anchor the heart valve prosthesis, the outer frame including a plurality of first crowns positioned around an outflow end of the outer frame and a plurality of second crowns positioned inward of the plurality of first crowns; and
a single-layer skirt disposed within and coupled to the outer frame and consisting of a single piece of material with minimal overlap of itself or another piece of skirt material when the heart valve prosthesis is in an expanded configuration.

2. The heart valve prosthesis of claim 1, wherein the skirt is coupled to the outer frame by at least one suture.

3. The heart valve prosthesis of claim 1, wherein the skirt further comprises a ring portion and a conical portion, ring portion having an inflow edge and being located at an inflow side of the heart valve prosthesis and the conical portion having an outflow edge and being located at an outflow side of the heart valve prosthesis.

4. The heart valve prosthesis of claim 3, wherein the inflow edge is configured with a plurality of slits.

5. The heart valve prosthesis of claim 3, wherein the conical portion is configured with a plurality of notches in the outflow edge.

6. The heart valve prosthesis of claim 5, wherein each of the plurality of notches is configured with a plurality of notch edges that, when sutured together, cause the conical portion to form a conical shape.

7. The heart valve prosthesis of claim 6, wherein, in a flat arrangement, the outflow edge is interrupted by the notches and, when the notch edges are sutured together, the outflow edge is continuous.

8. The heart valve prosthesis of claim 3, wherein, in a flat arrangement, the inflow edge is a first arc section of a first circle having a first diameter and the outflow edge is a second arc section of a second circle, concentric to the first circle, having a second diameter smaller than the first diameter.

9. The heart valve prosthesis of claim 8, wherein the ring portion includes ring connection edges disposed at ends of the inflow edge and the conical portion includes conical connection edges disposed at the ends of the outflow edge and intersecting the ring connection edges.

10. The heart valve prosthesis of claim 9, wherein the ring connection edges and the conical connection edges intersect at an angle.

11. The heart valve prosthesis of claim 9, wherein the ring connection edges are connected to the conical connection edges by an arcuate edge.

12. The heart valve prosthesis of claim 9, wherein the ring connection edges are collinear with the conical connection edges.

13. The heart valve prosthesis of claim 9, wherein the ring connection edges intersect the inflow edge at obtuse angles.

14. The heart valve prosthesis of claim 3, wherein the outflow edge has a length corresponding to a diameter of an outflow end of the outer skirt.

15. A method of assembling a heart valve prosthesis including an inner frame, an outer frame disposed around the inner frame, a valve component supported by the inner frame, and a skirt consisting of a single piece of material, the method comprising:
forming the skirt into a three-dimensional configuration by securing a plurality of connection edges to one another;
disposing the skirt within the outer frame, wherein a ring portion of the skirt including an inflow edge corresponds to a ring section of the outer frame and a conical portion of the skirt including an outflow edge corresponds to a tapered portion of the outer frame; and
coupling the skirt to the outer frame.

16. The method of claim 15, wherein the skirt is coupled to the outer frame by at least one suture.

17. The method of claim 15, wherein forming the skirt into the three-dimensional configuration includes securing together a plurality of opposing notch edges, the notch edges being configured to interrupt the outflow edge of the skirt.

18. The method of claim 15, wherein each of the plurality of connection edges includes ring connection edges disposed at ends of the ring portion and cone connection edges disposed at ends of the conical portion.

19. The method of claim 18, wherein the ring connection edges intersect the cone connection edges at an angle.

20. The method of claim 18, wherein an arcuate edge connects the ring connection edges to the cone connection edges.
